(19) United States
(12) Patent Application Publication
(10) Pub. No.: US 2023/0074207 A1
(43) Pub. Date: Mar. 9, 2023

(54) METHOD FOR DETERMINING KINETOSIS

(71) Applicant: ZF Friedrichshafen AG, Friedrichshafen (DE)

(72) Inventors: Mohamad Alayan, Tettnang (DE); Florian Dauth, Kressbronn (DE)

(73) Assignee: ZF Friedrichshafen AG, Friedrichshafen (DE)

(21) Appl. No.: 17/760,286

(22) PCT Filed: Jan. 22, 2021
(86) PCT No.: PCT/EP2021/051404

§ 371 Date: Aug. 5, 2022

(30) Foreign Application Priority Data
Feb. 6, 2020 (DE) 10 2020 201 442.6

(51) Int. Cl. A61B 5/00; G06V 40/10; G06V 20/59; A61B 5/11; G16H 40/63

(52) U.S. Cl. CPC A61B 5/4266; G06V 40/10; G06V 20/597; A61B 5/0082; A61B 5/1112; A61B 5/7278; G16H 40/63; A61B 2562/0219; A61B 2503/12

(57) ABSTRACT

The invention relates to a method for determining kinetosis in a vehicle user of a vehicle during at least one travel event in which at least one body part of the vehicle user is monitored, as a result of which image data are generated. Driving dynamics of the vehicle are monitored as the vehicle is being driven, as a result of which driving dynamics data are generated for every travel event while the vehicle is in motion. The image data are evaluated to determine the formation of sweat on the at least one body part of the vehicle user, as a result of which approximated electrodermal activity data are generated. The driving dynamics data are associated with the approximated electrodermal activity data, as a result of which the kinetosis of the vehicle user in at least one of the travel events is determined.

The present invention relates to a method for determining kinetosis, an evaluation device, a computer program product, and a vehicle.

A problem which is gaining more and more importance in journeys with a possibly automated vehicle is kinetosis, also referred to as motion sickness. Kinetosis can arise, for example, due to a sensory conflict. One presumes here that a vehicle user does not look at the route, but rather at his laptop, smart phone, inter alia, and thus his visual system communicates to the brain that it is in a stationary state. The vestibular system indicates precisely the contrary, however, due to externally acting accelerations on the body during the journey. These two contradictory items of information result in a so-called sensory mismatch in the vehicle user, which ultimately results in kinetosis. This results in comfort losses of the vehicle user, which is reflected in possible nonacceptance of the vehicle.

If one considers mobility of the future, it could be that the vehicle user enters his vehicle to drive to a destination. If this is a longer route, he will wish to relax on the freeway or the like and will have the vehicle drive autonomously. As soon as he relinquishes the control of the vehicle, he is no longer concentrating on the journey. This means that he will now also build up kinetosis more strongly. If there are also vehicle users on the backseats, he could also turn his seat around to converse with them. He now travels in reverse in addition, because of which his kinetosis should strengthen even more. Comfort losses thus arise, because symptoms such as nausea, vomiting, and headaches can occur.

Electrodermal activity (EDA) is often used to determine kinetosis. This is a short-term reduction of the conduction resistance of the skin. This effect is caused by emotional-affective reactions of the person and is noticeable due to the perspiration on specific body parts. Psychophysiological activities (for example emotions) of the person may be determined by measuring the EDA. The EDA can be determined by means of endosomatic measurement or also by means of exosomatic measurement. In the former, the electrical voltage of the skin is measured in that tiny electrodes are pierced into the skin to measure the activity of the nerves. In exosomatic measurement, a weak current is applied to the skin and kept constant. The EDA itself consists of two components: the tonic and phasic EDA. Both have properties, which can be clarified by the measurement of the skin conductivity. The tonic EDA can be viewed here as a long-term parameter, since the conductivity of the skin changes over a longer time period and is accordingly also measured. The skin conductivity is correlated with the body temperature of the person. The phasic EDA, in contrast thereto, is a short-term direct increase of the skin conductivity. It begins several seconds after corresponding stimulation and then disappears again rapidly.

A vehicle system is known from US 20190133511 A1, which comprises a seat in which a system for detecting kinetosis is integrated. The system for detecting kinetosis is configured so that it outputs signals detected from a vehicle occupant, e.g., motion, oscillations, physiological parameters, an electrodermal potential signal, and the like.

A method for reducing the motion sickness in a vehicle is known from EP 3303025 A4.

Proceeding from the prior art, the present invention is based on the object of providing an alternative method for determining kinetosis in a vehicle user which can be carried out in a contactless manner.

Proceeding from the stated object, the present invention proposes a method for determining kinetosis having the features according to claim 1, an evaluation device having the features according to claim 7, a computer program product having the features according to claim 8, and a vehicle having the features according to claim 9. Further advantageous embodiments and refinements are disclosed in the dependent claims.

In a method for determining kinetosis in a vehicle user of a vehicle during at least one travel event, at least one body part of the vehicle user is monitored, by which image data are generated. Driving dynamics of the vehicle are monitored during a journey of the vehicle, by which driving dynamics data are generated for each travel event during the journey. The image data are evaluated to ascertain a perspiration on the at least one body part of the vehicle user, by which approximated electrodermal activity data are generated. The driving dynamics data are linked to the approximated electrodermal activity data, by which the kinetosis of the vehicle user during at least one of the travel events is determined.

The vehicle can be designed for this purpose as a land vehicle, water vehicle, or air vehicle. The vehicle is preferably a land vehicle, e.g., a passenger vehicle, commercial vehicle, bus, people mover, or the like. The vehicle is formed in such a way that it can carry out automated functions. For example, automated functions of the vehicle can be autonomous or semi-autonomous driving. The vehicle can execute, for example, automated functions from level 3. Level 3 relates here to the autonomy level according to SAE J3016.

The vehicle user is a person who is located inside the passenger compartment of the vehicle and travels with the vehicle. The vehicle user can be oriented in the travel direction or in any other way.

The vehicle moves during its journey along a route. Multiple travel events can occur during the journey. At least one travel event always occurs during a journey. A travel event is defined as an event which deviates from a constant straight ahead journey. For example, a travel event of the vehicle can be cornering, braking, acceleration, evasion, merging out, merging in, or the like. Each travel event has certain vehicle dynamics, for example, a positive or negative acceleration and/or a set steering angle.

During the journey of the vehicle, the vehicle user, more precisely at least one body part of the vehicle user is monitored. This body part can be, for example, a face of the vehicle user, e.g., especially the forehead, the cheeks, or the upper lip, a hand of the vehicle user, e.g., especially the dominant or nondominant hand, a forearm of the vehicle user, a throat of the vehicle user, a chest area of the vehicle user, or a neck of the vehicle user. Of course, multiple body parts of the vehicle user can be monitored. The camera is configured here in such a way that it can acquire a reflection of the skin at the corresponding at least one body part of the vehicle user and a change of the same reflection. The monitoring of the at least one body part is preferably carried out by means of at least one camera. This camera is arranged in the interior of the vehicle and is placed so that it can monitor the at least one body part of the vehicle user. For example, this camera can be arranged in or above the head height of the vehicle user.

Image data are generated by means of the camera by the monitoring of the at least one body part of the vehicle user. These image data are subsequently evaluated by an evaluation device of the vehicle. For this purpose, the camera is connected to the evaluation device, so that a data and signal exchange can take place. The connection can be made wireless or wired. Starting from the acquired reflection of the skin on the at least one body part of the vehicle user, a perspiration on this body part is ascertained by means of the evaluation device. The perspiration on the at least one body part is directly correlated with the reflection of the skin on the same body part. If the perspiration is strongly pronounced, the reflection is stronger than in the case of a weak perspiration. For example, the evaluation device can make use of a trained artificial neural network to evaluate the image data with respect to the perspiration.

Proceeding from these data on the perspiration, approximated electrodermal activity data (EDA) are generated, because stronger perspiration is correlated with an elevated EDA, thus an elevated skin conductivity. Weaker perspiration is correlated with a low EDA, thus a low skin conductivity. If the at least one body part thus has a strong reflection, the approximated EDA is high and kinetosis has occurred. If the at least one body part thus has a low reflection, the approximated EDA is low and kinetosis has not occurred. The generation of the approximated EDA preferably takes place starting from a database in which electrodermal activity data are linked to data on perspiration.

During the journey of the vehicle, the driving dynamics of the vehicle are monitored, by which driving dynamics data are generated for each travel event during the journey. In other words, the driving dynamics are changed or adapted on the basis of the respective travel events. Driving dynamics are to be understood here as longitudinal dynamics, thus a positive or negative acceleration of the vehicle, transverse dynamics, thus setting a steering angle, and a rotational movement, thus pitching, yawing, and rolling. It is thus monitored whether and how strongly the vehicle accelerates, decelerates, turns, pitches, rolls, and/or yaws. The driving dynamics data thus comprise data on the acceleration, on the steering angle, on pitching movements, on yawing movements, and/or on rolling movements.

The driving dynamics are monitored by means of one driving dynamics sensor or by means of multiple driving dynamics sensors. The driving dynamics sensor is connected to the evaluation device, so that a data and signal exchange can take place. The connection can be made wireless or wired. The driving dynamics sensor can be formed, for example, as an acceleration sensor and/or as a gyro sensor. Alternatively thereto, the driving dynamics sensor can be designed as a velocity sensor, as an inclination sensor, as a steering angle sensor, as a yaw rate sensor, or as another suitable sensor.

The driving dynamics data are linked to the approximated EDA, by which the kinetosis of the vehicle user is determined during at least one of the travel events. It can thus be ascertained which travel events trigger kinetosis in the vehicle user. It can thus be determined whether and to what extent acceleration, deceleration, turning, pitching, rolling, or yawing, which are all related to certain travel events, trigger kinetosis in the vehicle user.

The method presented here is advantageous in that the vehicle user does not have to touch a sensor to be monitored with respect to kinetosis. The vehicle user can thus take his hands from the steering wheel, position his seat differently, and the like without having to accept losses in the ascertainment of kinetosis. Moreover, the measurement becomes more comfortable for the vehicle user, since he no longer perceives that measurement data are sampled.

According to one refining embodiment, if kinetosis is determined during the at least one travel event, the at least one travel event is stored as kinetosis-triggering. For example, the respective travel event is stored in a central memory of the vehicle and/or in an external memory, for example, in a cloud, as kinetosis-triggering. If kinetosis is thus determined during a travel event, this is stored with its corresponding driving dynamics data in the memory. It is thus possible for the vehicle user to identify and store individual kinetosis-triggering events.

According to one refining embodiment, during a repetition of the at least one travel event or during a new travel event which is comparable to the at least one travel event, the driving dynamics of the vehicle are adjusted so that reduced kinetosis or no kinetosis is triggered. A travel event which is comparable to the at least one travel event has identical or very similar driving dynamics as the at least one travel event. The driving dynamics are adjusted in such a way that reduced kinetosis or no kinetosis is triggered. That is to say that, for example, a lower positive or negative acceleration is selected, or that softer turning takes place. Additionally, further measures can be taken to counteract the kinetosis, for example, supplying fresh air into the vehicle, adjusting the temperature in the vehicle, changing the lighting in the vehicle, adjusting a seat alignment, or the like.

For this purpose, the evaluation device of the vehicle can be connected to further vehicle systems, so that a data and signal exchange can take place. For example, the evaluation device can be connected to a steering system, to a braking system, to a drive system, to an air-conditioning system, to an interior lighting system, to a seat controller, or the like. The evaluation device can activate, for example, the above-mentioned systems so that they can be adjusted.

The evaluation device for the vehicle is configured to be connected to the camera of the vehicle and to the driving dynamics sensor of the vehicle, wherein the evaluation device has means to carry out the method which was already described in the above description. That is to say, the evaluation device is connected to the above-mentioned systems and devices of the vehicle when the evaluation device is used in a vehicle. The evaluation device, the driving dynamics sensor, and the camera were already described in the above description. The connection of the evaluation device to the above-mentioned devices of the vehicle was also already described in the above description.

The evaluation device has means to carry out methods which were already described in the above description. These means can be designed, for example, as a computer program product which runs on the evaluation device. The method was already described. In addition, the evaluation device can activate the steering system and/or the braking system and/or the drive system so that the driving dynamics are adjusted, as already described.

The computer program product comprises commands which, upon execution of the program by the above-described evaluation device, carry out the method which has also already been described. The computer program product can comprise program code which contains these commands. The program code can be embodied, for example, on a data carrier or as a downloadable data stream.

The vehicle has the evaluation device which was already described. Moreover, the vehicle has the camera and the driving dynamics sensor which were already described. The evaluation device is connected to the camera and the driving dynamics sensor. The vehicle is configured to carry out automated functions, for example, to drive autonomously. This was already described.

Various exemplary embodiments and details of the invention are described in more detail on the basis of the figures explained hereinafter. In the figures:

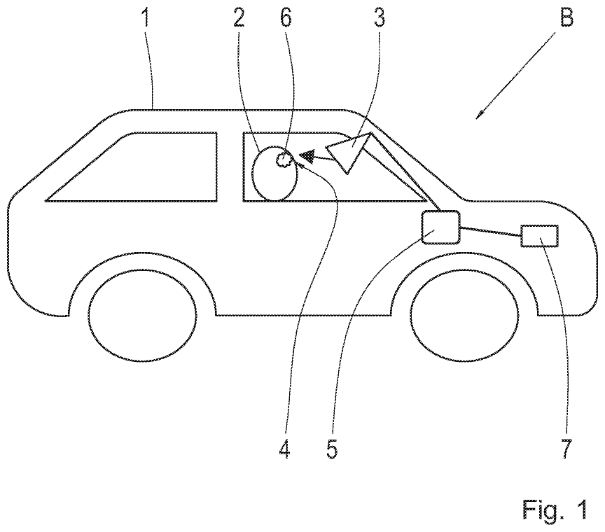

FIG. 1 shows a schematic illustration of a vehicle according to one exemplary embodiment,

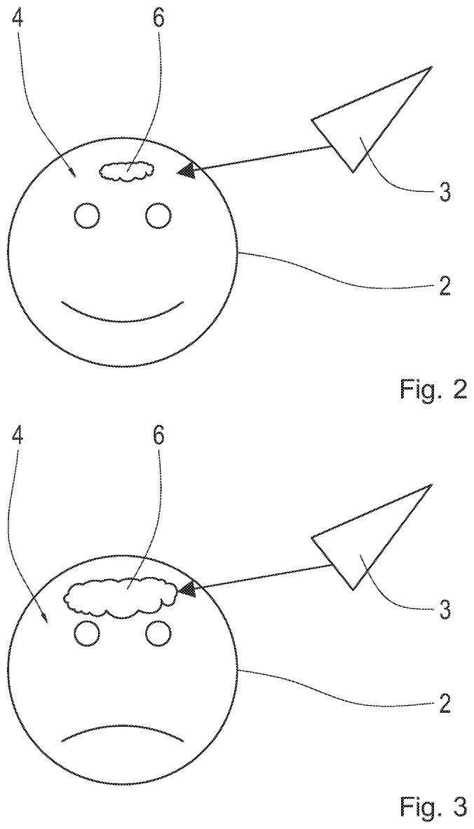

FIG. 2 shows a schematic illustration of a forehead of the vehicle user of the vehicle from FIG. 1 without kinetosis,

FIG. 3 shows a schematic illustration of the forehead of the vehicle user from FIG. 2 with kinetosis,

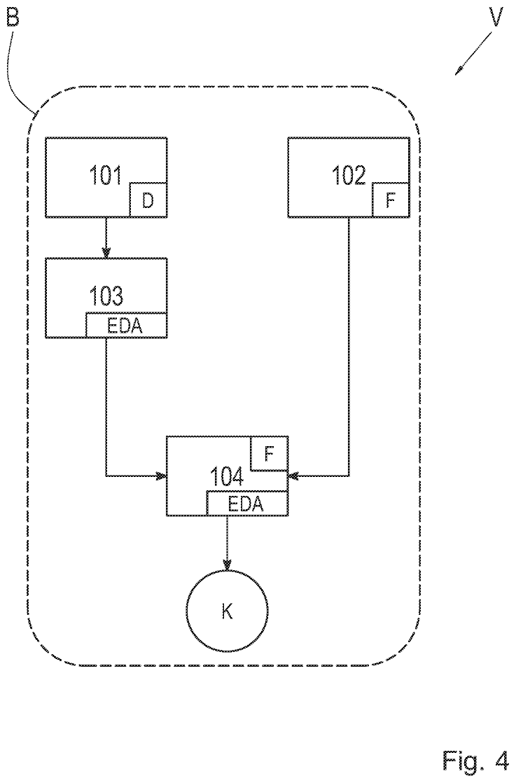

FIG. 4 shows a schematic illustration of a method which is carried out by the vehicle from FIG. 1.

FIG. 1 shows a schematic illustration of a vehicle 1 according to one exemplary embodiment. In the vehicle 1, a vehicle user 2 is located in the passenger compartment, wherein the vehicle user 2 travels with the vehicle 1. The vehicle 1 is capable of carrying out automated functions, for example, driving autonomously. The vehicle 1 travels along a route, wherein during this journey a travel event B occurs. This travel event B can be, for example, cornering or an acceleration.

The vehicle 1 has an evaluation device 5, a camera 3, and a driving dynamics sensor 7. The evaluation device 5 is connected to the camera 3, so that a data and signal exchange can take place. The connection can be made wireless or wired. The evaluation device 5 is connected to the driving dynamics sensor 7, so that a data and signal exchange can take place. The connection can be made wireless or wired.

The camera 3 is arranged in the vehicle interior, more precisely in the passenger compartment of the vehicle 1. The camera 3 is aligned and arranged so that it can at least partially acquire the vehicle user 2. The camera 3 thus acquires a body part 4 of the vehicle user 2, the forehead here. The body part 4 of the vehicle user 2 is monitored by means of the camera 3 for reflections, which are caused by perspiration 6 on the forehead of the vehicle user 2. Image data, which are evaluated by the evaluation device 5, are generated by the monitoring by means of the camera 3. The image data are therefore passed on to the evaluation device 5.

The evaluation device 5 evaluates the image data of the camera 3 to infer the strength of the perspiration 6. Strongly acquired reflections are evaluated by the evaluation device 5 as strong perspiration 6 on the body part 4. Weakly acquired reflections are evaluated by the evaluation device 5 as weak or nonexistent perspiration 6 on the body part 4. For example, the evaluation device 5 can make use of a trained artificial neural network to evaluate the image data with respect to the perspiration. The evaluation device 5 approximates the electrodermal activity data EDA starting from the determined perspiration 6 or the degree of the determined perspiration 6. This is carried out, for example, starting from a database in which electrodermal activity data EDA are linked to data on the perspiration 6. If the approximated electrodermal activity data EDA are high, this means that the vehicle user 2 has symptoms of kinetosis. If the approximated electrodermal activity data EDA are low, this means that the vehicle user 2 has no symptoms of kinetosis.

The driving dynamics sensor 7, which can be formed, for example, as an acceleration sensor or as a gyro sensor, ascertains the driving dynamics for each travel event B during the journey of the vehicle 1, by which driving dynamics data are generated. For example, the driving dynamics sensor 7 ascertains the positive and/or the negative acceleration during the travel event B. The ascertained driving dynamics data are passed on to the evaluation device 5.

The evaluation device 5 subsequently links the driving dynamics data to the approximated electrodermal activity data EDA. The kinetosis of the vehicle user 2 during the travel event B is thus determined. It can thus be ascertained which travel events B trigger kinetosis in the vehicle user 2. It can thus be determined whether and to what extent acceleration, braking, turning, pitching, rolling, or yawing, which are all related to specific travel events B, trigger kinetosis in the vehicle user 2.

FIG. 2 shows a schematic illustration of a forehead of the vehicle user 2 of the vehicle 1 from FIG. 1 without kinetosis. The forehead of the vehicle user 2 represents the body part 4 which is monitored by means of the camera 3. Alternatively thereto, instead of the forehead or simultaneously thereto, hands of the vehicle user 2, cheeks of the vehicle user 2, a neck of the vehicle user 2, an upper lip of the vehicle user 2, a throat of the vehicle user 2, a chest area of the vehicle user 2, or the like could be monitored as the body part 4.

It can be seen clearly here that the forehead of the vehicle user 2 only has minor perspiration 6 during the present travel event. This minor perspiration 6 only causes a slight reflection, which is acquired by the camera 3. Therefore, low electrodermal activity data EDA are approximated by the evaluation device. The vehicle user 2 thus does not have kinetosis.

FIG. 3 shows a schematic illustration of the forehead of the vehicle user 2 from FIG. 2 with kinetosis. The same vehicle user 2 is shown here as in FIG. 2. The forehead of the vehicle user 2 again represents the body part 4 which is monitored by means of the camera 3. It can be seen clearly here that the forehead of the vehicle user 2 has strong perspiration 6 during the present travel event. This strong perspiration 6 causes a strong reflection, which is acquired by the camera 3. High electrodermal activity data EDA are thus approximated by the evaluation device. The vehicle user 2 thus has kinetosis.

FIG. 4 shows a schematic illustration of a method V which is executed by the vehicle from FIG. 1. The method V runs during the journey of the vehicle during a travel event B.

In a first step 101, the body part of the vehicle user is monitored by means of the camera, by which image data D are generated. These image data D comprise data on the reflection of the body part of the vehicle user.

In a second step, which runs in parallel, for example, driving dynamics of the vehicle are monitored during a journey of the vehicle by means of the driving dynamics sensor, by which driving dynamics data F are generated for the travel event B.

In a third step 103 following first step 101, the image data D are evaluated by means of the evaluation device to ascertain perspiration on the body part of the vehicle user, by which approximated electrodermal activity data EDA are generated. The perspiration is ascertained starting from the acquired reflection of the body part. For this purpose, the evaluation device can make use, for example, of a trained artificial neural network. Third step 103 can run in parallel to second step 102, for example. The electrodermal activity data EDA are approximated starting from the strength of the perspiration. A database can be used for this purpose, for example, in which various electrodermal activity data EDA are linked to data on the perspiration or degrees of perspiration.

In a last fourth step 104, the driving dynamics data F are linked to the approximated electrodermal activity data EDA, by which the kinetosis K of the vehicle user during the travel events B is determined. It can thus be ascertained which travel events B trigger kinetosis K in the vehicle user.

The method V runs continuously during the journey of the vehicle. This means that the method V is continued when a travel event B is ended, but the vehicle moves further, or when kinetosis K has been determined in the vehicle user.

The examples shown here are only selected by way of example. For example, another body part of the vehicle user or multiple body parts of the vehicle user can be monitored simultaneously by means of the camera. For example, more than one vehicle user can use the vehicle. In this case, all vehicle users are monitored to be able to determine kinetosis in them.

### LIST OF REFERENCE SIGNS

1 vehicle
2 vehicle user
3 camera
4 body part
5 evaluation device
6 perspiration
7 driving dynamics sensor
101 first step
102 second step
103 third step
104 fourth step
B travel event
D image data
EDA electrodermal activity data
F driving dynamics data
K kinetosis
V method

1. A method for determining kinetosis in a vehicle user of a vehicle during at least one travel event, wherein
at least one body part of the vehicle user is monitored, by which image data are generated,
driving dynamics of the vehicle are monitored during a journey of the vehicle, by which driving dynamics data are generated for each travel event during the journey,
the image data are evaluated to ascertain perspiration on the at least one body part of the vehicle user, by which approximated electrodermal activity data are generated,
the driving dynamics data are linked to the approximated electrodermal activity data, by which the kinetosis of the vehicle user during at least one of the travel events is determined.

2. The method as claimed in claim 1, wherein the monitoring of the at least one body part is carried out by means of a camera.

3. The method as claimed in claim 1, wherein the driving dynamics of the vehicle are monitored by means of a driving dynamics sensor.

4. The method as claimed in claim 3, wherein the driving dynamics sensor is formed as an acceleration sensor and/or as a gyro sensor.

5. The method as claimed in claim 1, wherein if kinetosis is determined during the at least one travel event, the at least one travel event is stored as kinetosis-triggering.

6. The method as claimed in claim 5, wherein during a repetition of the at least one travel event or during a new travel event, which is comparable to the at least one travel event, the driving dynamics of the vehicle are adapted, so that reduced kinetosis or no kinetosis is triggered.

7. An evaluation device for a vehicle, wherein the evaluation device is configured to be connected to a camera of the vehicle and to a driving dynamics sensor of the vehicle, wherein the evaluation device has means to carry out the method as claimed in any one of the preceding claims.

8. A computer program product comprising commands which, upon execution of the program by an evaluation device as claimed in claim 7.

9. A vehicle having an evaluation device as claimed in claim 7, a camera, and a driving dynamics sensor, wherein the evaluation device is connected to the camera and the driving dynamics sensor, wherein the vehicle is configured to carry out automated functions.
